(19) United States
(12) Patent Application Publication
(10) Pub. No.: US 2021/0244268 A1
(43) Pub. Date: Aug. 12, 2021

(54) TESTING A SUBJECT'S VISION

(71) Applicant: Azul Optics Limited, Bristol (GB)

(72) Inventors: Matthew John Evans, Bristol (GB); Shelby Eric Temple, Bristol (GB)

(73) Assignee: Azul Optics Limited, Bristol (GB)

(21) Appl. No.: 17/049,429

(22) PCT Filed: Apr. 23, 2019
(86) PCT No.: PCT/GB2019/051121

§ 371 Date: Oct. 21, 2020

(30) Foreign Application Priority Data
Apr. 26, 2018 (GB) 1806829.6

(51) Int. Cl. A61B 3/06; A61B 3/00

(52) U.S. Cl. CPC A61B 3/06; A61B 3/0008

(57) ABSTRACT

An apparatus (100, 200, 300, 400) for testing a subject's vision and a method of reducing intensity artefacts during a test of a subject's eye (109) are provided. The apparatus comprises: a light source arrangement (102) located towards a source end (104) of the apparatus, and arranged to produce polarized light; and one or more panels (108a, 108b, 108c), the one or more panels comprising one or more blocking regions that do not transmit polarized light, and defining one or more light transmitting regions (111), the one or more light transmitting regions (111) being just large enough to allow unobscured vision by the subject's eye of polarized light travelling directly from the light source arrangement when the subject's eye is positioned adjacent to a viewing end (106) of the apparatus, the one or more panels being located between the light source arrangement and the subject's eye.

### TECHNICAL FIELD

The present disclosure relates to the fields of optometry and ophthalmology. More particularly it relates to reducing intensity artefacts in the eye when viewing polarized light.

### BACKGROUND

Humans can perceive the polarization of light, although we are typically unaware of our ability to do so. When presenting polarized light stimuli for observation it is possible to inadvertently create intensity artefacts that vary in relative intensity as the angle of polarization is varied. These intensity artefacts can be of sufficiently high contrast to be detected during observation, thereby obviating attempts to determine if the observer can see only the changes due to a polarized light stimulus, for example, to run a test on the subject's visual function or decrease in visual function due to disease.

Such intensity artefacts are created when polarized light is reflected from surfaces between the observer and the polarized light stimulus. The magnitude of the intensity artefact is governed by specific variables, primarily: the angle of the surface relative to the optical axis between the observer's eye and the stimulus, with the greatest artefacts occurring at or near Brewster's angle; the differences in refractive index between the material that the surface is made of and the media through which the light is travelling, typically air, water, glass or transparent plastics, with the greatest artefacts occurring when the difference in index of refraction between the two materials is large; the surface texture of the reflecting material, with the greatest artefacts occurring when the surface is glossy/smooth and least significant artefacts occurring when the surface is highly rugose/irregular, which creates diffuse reflection; and the interaction between diffuse reflectance (albedo) of the reflecting surface and background/environmental light, with greatest artefacts occurring when the reflectance is low (e.g. dark materials like black plastic) with no background light, and least when the reflectance of the surface is high (e.g. diffuse white) and the background light intensity is high and matching the spectral quality (wavelength range and spectral peaks) of the spectral quality of the polarized stimulus.

Previous experiments and tests that use polarized light stimuli for testing human vision do not take these intensity artefacts into account, and thus run the risk of allowing such artefacts to interfere with the experiment or test. There therefore exists a need for a way of removing or reducing the intensity artefacts occurring in the observer's vision during the observation of polarized light.

### SUMMARY

In at least some embodiments, the present techniques provide an apparatus for testing a subject's eyes comprising: a light source arrangement located towards a source end of the apparatus, and arranged to produce polarized light; and one or more panels, the one or more panels comprising one or more blocking regions that do not transmit polarized light, and defining one or more light transmitting regions and further defining at least one edge at each light transmitting region, the one or more light transmitting regions being just large enough to allow unobscured vision by the subject's eye of polarized light travelling directly from the light source arrangement when the subject's eye is positioned adjacent to a viewing end of the apparatus, the one or more panels being located between the light source arrangement and the subject's eye. Such an apparatus is configured to reduce the intensity, salience or contrast of intensity artefacts to a level below detection by the subject. The use of positioned light transmitting regions constrains viewing angles for vision by the subject's eye. The light source arrangement may be an immediate source of polarized light, or it may produce unpolarized light that is polarized by a polarizing filter, for example.

The apparatus may comprise illumination means for illuminating at least one of the edges of the light transmitting regions, defined by the blocking regions. The at least one of the edges may include, in particular, the edge or edges located closest to the viewing end of the apparatus, or to the subject's eye.

The apparatus may comprise illumination means for illuminating regions of the subject's face other than the eyes. Such regions of the subject's face may include the nose, parts of the cheek(s), eyelashes, or eyelids, for example.

Such illumination means reduce intensity artefacts by adding background light to surfaces that cannot normally be obscured or removed from the subject's field of view, such as facial features and the edges of the light transmitting region, thereby minimising the difference in intensity between the viewed polarized light and the reflections of the polarized light from these surfaces.

The illumination means for illuminating at least one of the edges and for illuminating regions of the subject's face other than the eyes may be the same illumination means. Alternatively, the means for illuminating at least one of the edges and for illuminating regions of the subject's face other than the eyes may originate from different illumination means.

The illumination means may be formed in at least one of the panels, and may be configured to illuminate at least regions of the subject's face other than the eyes. Illumination means formed in at least one of the panels may alternatively be configured to illuminate at least one of the edges.

The illumination means may be configured to produce diffuse light.

The light transmitting regions of at least two panels may be aligned to allow unobscured vision by the subject's eye of polarized light travelling directly from the light source arrangement. In this instance, the light transmitting regions may be formed in or by the at least two panels.

The apparatus may comprise a tube, the tube supporting the one or more panels, the tube and the panels together defining one or more chambers. The tube may enable the apparatus to be presented in the form of a portable, unitary viewing apparatus. The tube may have any number of sides, and may be any generally extended shape, such as a prism, oblong or cylinder.

The tubular apparatus may further comprise at least two chambers and the illumination means may be arranged to illuminate at least or only the chamber closest to the viewing end. Such illumination enables the advantageous illumination of the edge or edges of the light transmitting region closest to the viewing end, which, if left unilluminated, may give rise to undesirable intensity artefacts.

The apparatus may further comprise an anti-reflective surface inside the tube.

The panel closest to the viewing end may be translucent.

At least one of the panels may be provided with a frosted region. In particular, the panel located closest to the subject's eye, or to the viewing end of the apparatus may be provided with a frosted region.

At least one of the panels may be angled with respect to the normal of a line between the light source arrangement and the subject's eye. “The line between the light source arrangement and the subject's eye” refers to a straight line between the light source arrangement and the subject's eye. The term “angled” refers to any non-zero angle, such that the plane or equivalently the general plane of the panel is not aligned along the normal of the line in question.

The edge of each light transmitting region may be sufficiently sharp to reduce or minimise the polarized light reflecting from the edge into the subject's eye. That is to say, the edges should not have rounded corners, and should instead be sharp acute angles. This minimises the area of surfaces that are present between the observer and the polarized light source, thus minimising the areas where intensity artefacts can occur.

The light transmitting regions may be apertures. Apertures refer to cut out portions of a solid object, such that there is an open channel or conduit between two sides of the solid object. The solid objects in this case are the panels.

The light source arrangement may be configured to produce light including a wavelength range of 400 nm to 550 nm.

The light source arrangement may be configured to generate full spectrum light.

The illumination means may be configured to produce light having a spectral composition that is substantially the same as the spectral composition of the output of the light source arrangement.

The apparatus may further comprise a platform for receiving at least a portion of the subject's face at the viewing end of the apparatus. Such a platform may be a rest for receiving the subject's face, chin, or forehead, or alternatively or additionally an eyepiece, for receiving the portion of the subject's face surrounding the eye. Such a platform may assist in aligning the subject's eye with the apparatus, in order that they are able to view the polarized light stimulus, whilst experiencing a lighting environment that reduces or removes intensity artefacts.

The apparatus may be adapted monocularly. By monocularly, it is meant that the apparatus enables one eye to be tested at a time.

The apparatus may be adapted binocularly. By binocularly, it is meant that the apparatus enables both eyes to be tested at the same time.

At least one of the light transmitting regions may be at or adjacent the viewing end of the apparatus.

According to a further aspect of the present invention, a method of reducing intensity artefacts during a test of a subject's eyes is provided, the method comprising: positioning the subject's eye adjacent to a light transmitting region; and directing polarized light through the light transmitting region for observation by the subject; wherein the light transmitting region is just large enough to allow unobscured vision by the subject's eye of polarized light travelling directly from a light source arrangement.

The method may further comprise illuminating regions of the subject's face other than the eyes.

The method may further comprise illuminating at least one of the light transmitting regions.

The embodiments described here may be used alongside experiments or tests that determine a subject's density of macular pigments; and/or their stage of various eye diseases such as age-related macular degeneration, diabetic retinopathy, amblyopia, macular edema, based on whether they can observe the Haidinger's brushes phenomenon. They may also be used to complement experiments involving Boehm's brushes.

### BRIEF DESCRIPTION OF THE DRAWINGS

The present techniques will be described further, by way of example only, with reference to embodiments thereof as illustrated in the accompanying drawings, in which:

FIG. 1 schematically illustrates an apparatus in one example embodiment in which light transmitting regions and illumination means are provided to reduce below a detection threshold, or to eliminate, intensity artefacts;

FIG. 2 schematically illustrates an apparatus in a further example embodiment in which light transmitting regions and illumination means are provided to reduce below a detection threshold, or to eliminate, intensity artefacts;

FIG. 3 schematically illustrates an apparatus in a further example embodiment in which light transmitting regions and illumination means are provided to reduce below a detection threshold, or to eliminate, intensity artefacts; and

FIG. 4 schematically illustrates an apparatus in a further example embodiment in which light transmitting regions are provided to reduce below a detection threshold, or to eliminate, intensity artefacts.

### DESCRIPTION OF EMBODIMENTS

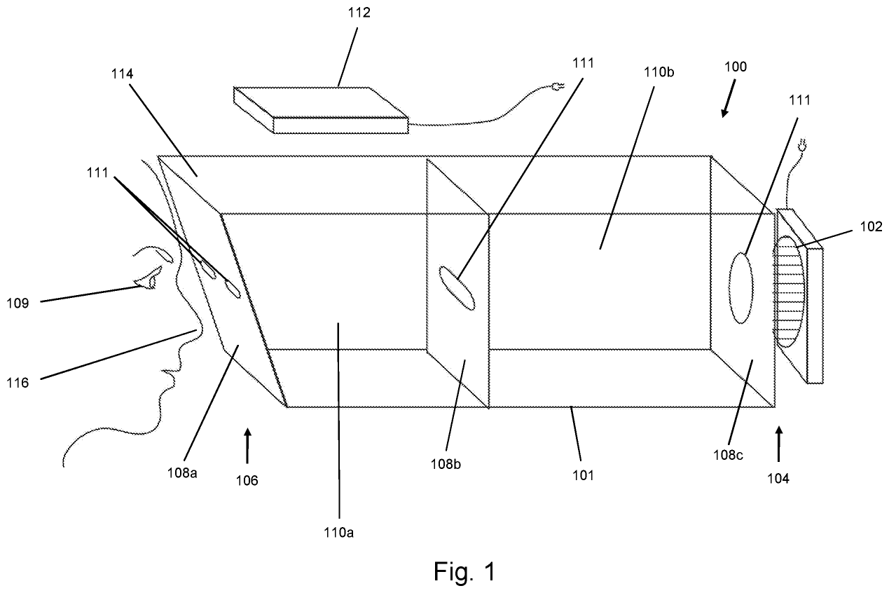

FIG. 1 schematically illustrates an example embodiment of an apparatus 100 for testing a subject's vision. The apparatus comprises a tubular arrangement 101. A polarized light source 102 that produces polarized light is located at a first end of the tubular arrangement 101, hereinafter referred to as the source end 104. The polarization light source 102 is illustrated as being an emitter of polarized light which draws power from an external power source. However, it will be appreciated that it may equally draw power from a self-contained power source. Similarly, the polarized light source 102 may be made up of a regular, unpolarized light source, the light from which is passed through a polarizing filter. The polarized light source 102 may be configured to produce light including a wavelength range of 400 nm to 550 nm. The polarized light source 102 may alternatively or additionally be configured to generated full spectrum light.

The tubular arrangement 101 further comprises a viewing end 106, located at the opposite end of the tubular arrangement 101 to the source end 104. In addition to these two ends, the tubular arrangement 101 comprises four side such that it defines a general box shape, and a number of panels 108a, 108b, 108c spaced along its length, the panels being supported by the sides of the tubular arrangement 101. Three panels 108a, 108b, 108c are illustrated, but a greater or fewer number may equally be used. In the present instance, the three panels 108a, 108b, 108c define two chambers 110a, 110b within the tubular arrangement 101. The panels 108a, 108b, 108c are each formed with at least one light transmitting region 111 therein. The light transmitting regions 111 are aligned with one another, and with the polarization light source 102, such that polarized light produced by the polarization light source 102 may be directed down the tubular arrangement from the source end 104 to the viewing end 106. The light transmitting regions 111 are just large enough to allow unobscured vision by a subject's eye 109 of the polarized light travelling directly from the polarized light source 102 when the subject's eye 109 is positioned adjacent to the viewing end 106. By directly, it is meant that light travels to the eye 109 from the polarized light source 102 and does not reflect from, or travel via, any other surfaces in-between. One or more of the panels 108a, 108b, 108c may be translucent or opaque, whilst the light transmitting regions 111 may be transparent regions of the panel, lenses, or cut out as apertures within the panel. Alternatively, one or more of the panels 108a, 108b, 108c may be frosted panels, with the light transmitting regions 111 being transparent regions of the panel, lenses, or cut out as apertures within the panel. An embodiment in which the panel 108a closest to the viewing end 106 is translucent or frosted is particularly advantageous for allowing additional illumination to be directed onto the subject's face, other than their eyes, when the subject's eye 109 is placed adjacent to the viewing end 106, as will be discussed in greater detail below. In the case where the light transmitting regions 111 are cut outs, apertures or any other form of “free space” in or defined by the panels 108a, 108b, 108c, it is advantageous for the edges of the panels 108a, 108b, 108c that define the cut outs, apertures or other form of “free space” to have sharp edges. That is to say, the edges should have acute angles with surfaces that do not sit at an angle that can reflect light from the polarized light source 102 into the subject's eye 109. This minimises the area of surfaces that is present between the observer and the polarization light source 102, thus minimising the effects of intensity artefacts, the causes of which will be discussed more below.

Illumination means 112 are positioned adjacent to the chamber 110a of the tubular arrangement 101 located closest to the viewing end 106. The illumination means 112 are arranged to produce unpolarized illumination. As with the polarization light source 102, the illumination means 112 are illustrated as drawing power from an external power source, but they could equally draw power from a self-contained power source. The side of the chamber 114 adjacent to the illumination means 112 is made of a transparent or translucent material, to allow the illumination produced by the illumination means 112 to be directed into the chamber 110a closest to the viewing end 106. The intensity of light emitted and the position of the illumination means 112 should be sufficient to project illumination onto panel 108a such that it transmits light, with substantially similar spectral quality to the polarized light source 102, onto the observers face 116. Alternatively, the side of the chamber 114 adjacent to the illumination means 112 may be removed altogether, to allow the illumination to illuminate the chamber 114.

A subject viewing polarized light would ordinarily experience intensity artefacts in their vision. However, a subject viewing polarized light through the apparatus 100 described herein will experience intensity artefacts with a reduced salience or intensity contrast, or possibly no intensity artefacts whatsoever, if the level of the intensity artefacts is below the subject's detection threshold. This is due to two effects implemented throughout the present invention: 1) restriction of viewing angles and 2) projection of light onto any surface that might reflect the polarized stimulus light into the field of view of the observer.

As mentioned previously, intensity artefacts arise due to reflection of polarized light from surfaces between the observer and the polarized light stimulus. In the present case, such surfaces may include the inner sides of the tubular arrangement 101, for example. Such surfaces may be blocked out from the vision of the observer through the combination of the panels 108a, 108b, 108c and the light transmitting regions 111, which only allow polarized light travelling directly from the polarized light source 102 to enter the subject's eye 109, and not polarized light that has been reflected via an inner surface of the tubular arrangement 101. At the very least, the polarized light travelling into the subject's eye that has been reflected from a surface between the observer and the polarized light source 102 is reduced, if not removed altogether.

The second effect is beneficial because the herein described surfaces between the observer and the polarized light source 102 are not limited to surfaces within the viewing apparatus. They also include surfaces or features 116 on the observer's face that are still visible by the observer, such as the nose, eyelashes and parts of the cheeks, for example. Ordinarily, projecting polarized light onto these features 116 would create intensity artefacts detectable by the observer. These can be reduced or eliminated completely by illuminating the features or surfaces 116 in question with an illuminating light source 112. It is beneficial for the illumination projected onto the features 116 to be of a similar spectral quality (i.e. similar relative intensity at each wavelength) to that of the polarized stimulus light being viewed. This is so that the relative intensity changes caused by the intensity artefacts are diluted by the added intensity of the illumination projected onto the facial features. This works because the intensity artefacts alone create a fluctuation in intensity as the angle of polarization changes (e.g. +3 units against a dark background of 0 units, which represents 100% change in intensity contrast) but this relative change is decreased when additional illumination is provided (e.g. when the background light intensity on the reflecting surface is increased from 0 to +500 units, the change in intensity of 3 units due to intensity artefacts, results in the +3 units becoming only a 0.6% change in intensity, which is below the threshold for detection). Accordingly, it is advantageous for the spectral composition of the illumination produced by the illumination means 112 to match the spectral composition of the polarized light produced by the polarized light source 102. In the presently-illustrated example, the illumination originates from the illumination light source 112, which illuminates the chamber 110a. In the case that the panel 108a closest to the viewing end 106 is translucent or frosted, or any other material with the effect of diffusing light, light from the illuminated chamber (originating from the illumination light source) is transmitted through the panel onto the subject's face, in particular onto features of the face that are not limited to the subject's eyes. The illumination impinging on the subject's face in this manner may be diffuse light, after travelling through the translucent, frosted or otherwise diffusing panel. In the case that the panel 108a closest to the viewing end 106 is opaque, the illumination light may pass through the chamber 110a, through the light transmitting regions 111 in panel 108a onto the subject's face, particularly the features of the face that are not limited to the subject's eyes. There may also or alternatively be a reflecting light source located on the same side as the observer, which projects illumination onto the subject's face and/or the edge(s) of the light transmitting regions.

In addition to illuminating the features 116 of the subject's face, as discussed above, it is also advantageous to illuminate the edges of the light transmitting region(s) 111 in the panel 108a closest to the viewing end 106, as these edges can also act as a surface from which intensity artefacts arise. In the illustrated example, including the alternatives in the construction of the panel 108a discussed above, this is achieved by the illumination of the chamber 110a, which allows illumination of the edges of light transmitting regions 111 in the panel 108a closest to the viewing end 106. Alternatively, an illumination light source, separate to that which illuminates the face, may be used.

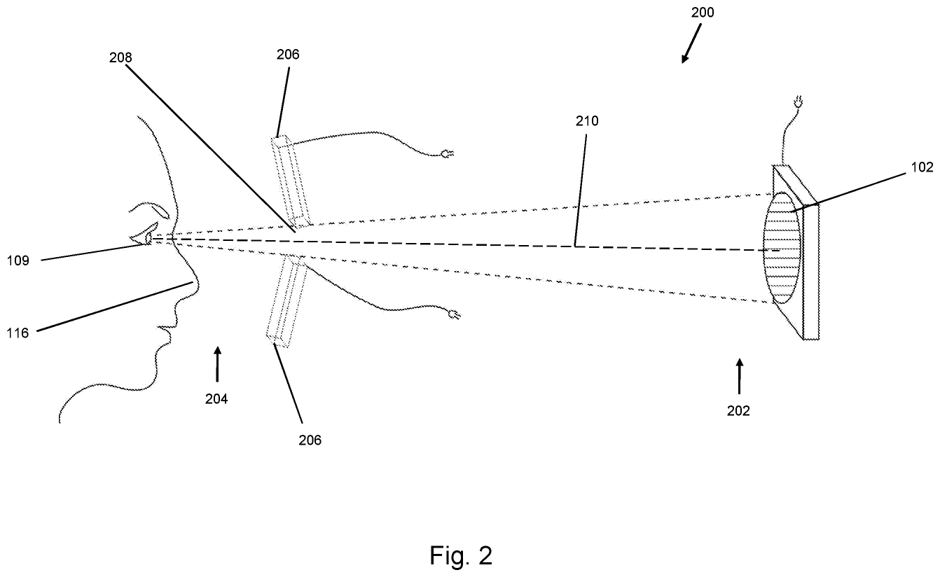

FIG. 2 shows an alternative embodiment of the present invention. An apparatus 200 is shown, the apparatus 200 having a polarization light source 102 present at a source end 202 of the apparatus 200. The apparatus 200 further comprises a viewing end 204. Between the source end 202 and the viewing end 204, there exists two blocking panels 206. The two blocking panels 206 define a light transmitting region 208, through which polarized light produced by the polarized light source 102 is able to travel. In the illustrated example, the light transmitting region 208 contains only free space, i.e. there is no solid material filling it in, however, it will be appreciated that the light transmitting region 208 may equally be a solid but transparent material or lens supported between the blocking panels 206. The blocking panels 206 limit the vision of surfaces between the observer's eye 109 and the polarized light source 102, in the same manner as the panels 108a, 108b, 108c. The blocking panels 206 are also oriented at an angle with respect to a normal of the line 210 between the observer's eye 109 and the polarization light source 102. Such angling offers the advantage that the surface of the edge of light transmitting region 208 from which intensity artefacts could arise is reduced or obscured, thereby minimising the intensity artefacts themselves.

In this embodiment, the blocking panels 206 also act as illumination light sources arranged to illuminate the features 116 of the subject's face, not limited to the eyes. As with the previous illumination light sources, these are illustrated as drawing their power from an external power source, but it will again be appreciated that an internal, portable or self-contained power source could also be used. Once again, the light transmitting region 208 may be illuminated by the illumination light sources of blocking panels 206, or alternatively by a separate, dedicated illumination light source.

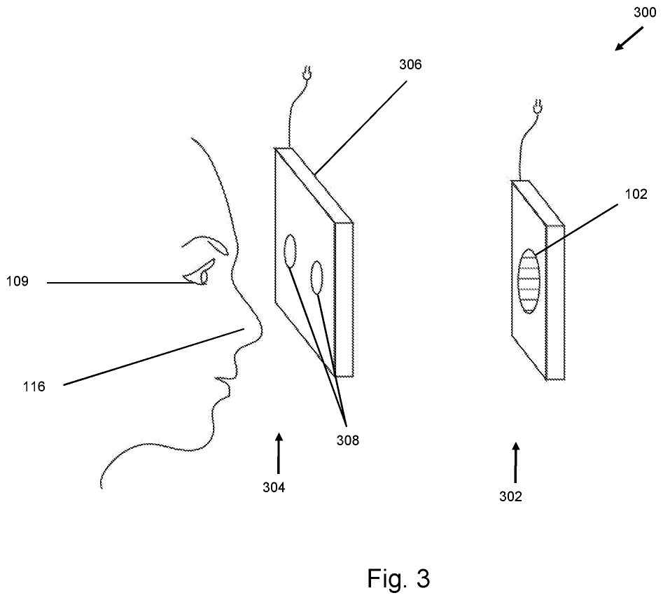

FIG. 3 shows an alternative embodiment according to the present invention. The apparatus 300 comprises a polarized light source 102 at the source end 302, as well as a viewing end 304. A panel 306 is located between the viewing end 304 and the source end 302, the panel 306 comprising two light transmitting regions 308, such that a subject's eye 109 positioned at the viewing end 304 of the apparatus 300 has an unobscured vision of the light travelling directly from the polarized light source 102. As with previously discussed embodiments, the light transmitting regions 308 may be apertures formed within the panel 306, or they may be some transparent material, such as glass mounted or formed in the panel, for example. There are two light transmitting regions 308 in the panel 306 of this embodiment, and as such, the apparatus 300 described herein is adapted binocularly. That is to say, it is possible to carry out the methods described herein on both of the subject's eyes simultaneously. However, it is also possible to perform the described methods on only one of the eyes at a time if so desired, and is thus also considered to be monocularly adapted. As with previously described embodiments, the light transmitting regions 308 are just large enough to allow unobscured vision by the subject's eye 109 of polarized light travelling directly from the polarized light source 102 when the subject's eye 109 is positioned adjacent to the viewing end 304.

The panel 306 again further comprises an illumination light source, configured to illuminate at least the subject's facial features 116, not limited to the eyes 109, as well as possibly the edges of the light transmitting regions 308 formed in this panel 306, that is, the panel positioned closest to the viewing end 304. Though, as mentioned previously, such illumination of the edges of the light transmitting regions 308 may be performed by a separate illumination light source.

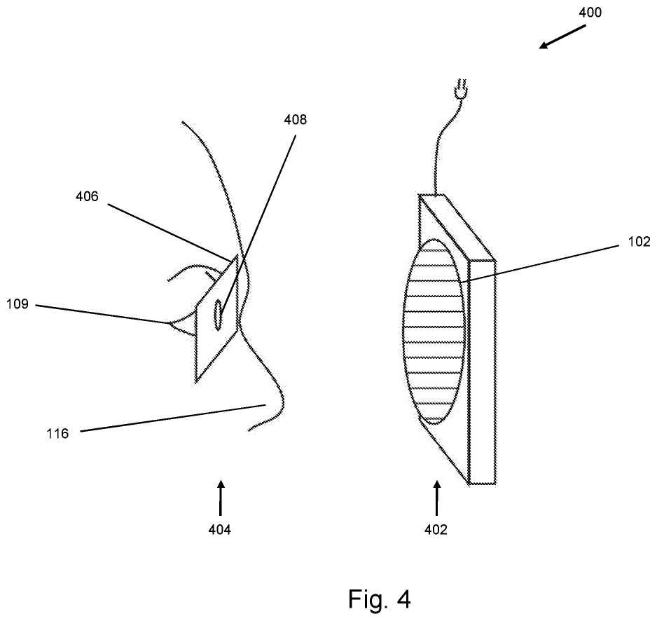

FIG. 4 illustrates yet a further embodiment of the present invention. As with previously described embodiments, the apparatus 400 comprises a source end 402, towards or at which is located a polarized light source 102, and a viewing end 404. In this embodiment, a panel 406 is located at the viewing end 404 of the apparatus 400. The panel 406 comprises a light transmitting region 408, which as previously discussed, may for example be an aperture or a solid but transparent material, for example. The panel 406 is positioned such that the light transmitting region 408 is aligned perfectly or close to perfectly with the pupil of the subject's eye 109, which is positioned at the viewing end 404. In this manner, surfaces between the observer's eye 109 and the polarization light source 102 are not visible to the subject. Due to the careful alignment of the light transmitting region 408 with the pupil of the subject's eye, such surfaces rendered not visible include the facial features 116 of the subject, which may normally contribute to the intensity artefacts experienced. There is therefore no requirement for further illumination means to illuminate these facial features 116. Further, no illumination means will be required to illuminate the light transmitting region 408, again due to the careful alignment of the light transmitting region 408 with the pupil of the subject's eye. The edge of the aperture 408 is an acute angle and forms a sharp edge thus reducing the surface area upon which intensity artefacts could be formed. The apparatus 400 described herein allows one eye to be tested at a time, and is hence monocularly adapted. Though it will also be appreciated that two such panels could be positioned, one on each eye, such that both eyes could be tested at once, in which case the apparatus would hence be binocularly adapted.

Fundamentally, the embodiments of the present invention aim to obscure or remove surfaces between the observer's eye and the polarized light stimulus, and to add background light to any surface that cannot be removed, such that the background light reduces the relative contrast of the change in intensity caused by the intensity artefacts to below the observer's threshold for detection.

In the present application, the words “configured to . . . ” or “arranged to” are used to mean that an element of an apparatus has a configuration able to carry out the defined operation. In this context, a “configuration” means an arrangement or manner of interconnection of hardware or software. For example, the apparatus may have dedicated hardware, which provides the defined operation, or a processor or other processing device may be programmed to perform the function. “Configured to” or “arranged to” does not imply that the apparatus element needs to be changed in any way in order to provide the defined operation.

Although illustrative embodiments have been described in detail herein with reference to the accompanying drawings, it is to be understood that the invention is not limited to those precise embodiments, and that various changes, additions and modifications can be effected therein by one skilled in the art without departing from the scope of the invention as defined by the appended claims. For example, various combinations of the features of the dependent claims could be made with the features of the independent claims without departing from the scope of the present invention.

1. Apparatus for testing a subject's eyes comprising:
a light source arrangement located towards a source end of the apparatus, and arranged to produce polarized light; and
one or more panels, the one or more panels comprising one or more blocking regions that do not transmit polarized light, and defining one or more light transmitting regions and an edge at each light transmitting region, the one or more light transmitting regions being just large enough to allow unobscured vision by the subject's eye of polarized light travelling directly from the light source arrangement when the subject's eye is positioned adjacent to a viewing end of the apparatus, the one or more panels being located between the light source arrangement and the subject's eye.

2. The apparatus as claimed in claim 1; comprising illumination means for illuminating at least one of the edges.

3. The apparatus as claimed in claim 1; comprising illumination means for illuminating regions of the subject's face other than the eyes.

4. The apparatus as claimed in claim 3; further comprising illumination means for illuminating at least one of the edges, wherein the illumination means for illuminating at least one of the edges and illuminating means for illuminating regions of the subject's face other than the eyes are the same illumination means.

5. The apparatus as claimed in claim 3; wherein the illumination means for illuminating regions of the subject's face other than the eyes is formed in at least one of the panels, and is configured to illuminate at least regions of the subject's face other than the eyes.

6. The apparatus as claimed in claim 3; wherein the illumination means for illuminating regions of the subject's face other than the eyes is configured to produce diffuse light.

7. The apparatus as claimed in claim 1 and having a plurality of the panels; wherein the light transmitting regions of at least two of the panels are aligned to allow unobscured vision by the subject's eye of polarized light travelling directly from the light source arrangement.

8. The apparatus as claimed in claim 1; further comprising a tube, the tube supporting the one or more panels, the tube and the one or more panels together defining one or more chambers.

9. (canceled)

10. (canceled)

11. The apparatus as claimed in claim 1; wherein the panel closest to the viewing end is translucent.

12. The apparatus as claimed in claim 1; wherein at least one of the panels is provided with a frosted region.

13. The apparatus as claimed in claim 1; wherein at least one of the panels is angled with respect to the normal of a line between the light source arrangement and the subject's eye.

14. The apparatus as claimed in claim 1; wherein the edges are sufficiently sharp to minimise the polarized light reflecting from the edge into the subject's eye.

15. The apparatus as claimed in claim 1; wherein the light transmitting regions are apertures.

16. The apparatus as claimed in claim 1; wherein the light source arrangement is configured to produce light including a wavelength range of 400 nm to 550 nm.

17. The apparatus as claimed in claim 1; wherein the light source arrangement is configured to generate full spectrum light.

18. The apparatus as claimed in claim 3, wherein the illumination means for illuminating regions of the subject's face other than the eyes is configured to produce light having a spectral composition that substantially matches that of the output of the light source arrangement.

19. (canceled)

20. (canceled)

21. The apparatus as claimed in claim 1; wherein at least one of the light transmitting regions is at or adjacent the viewing end of the apparatus.

22. A method of reducing intensity artefacts artifacts during a test of a subject's eyes comprising:
positioning the subject's eye adjacent to a light transmitting region; and
directing polarized light through the light transmitting region for observation by the subject; wherein the light transmitting region is just large enough to allow unobscured vision by the subject's eye of polarized light travelling directly from a light source arrangement.

23. The method as claimed in claim 22; further comprising illuminating regions of the subject's face other than the eyes.

24. The method as claimed in claim 22; further comprising illuminating at least one edge of the light transmitting regions.
